(19) United States
(12) Patent Application Publication
(10) Pub. No.: US 2022/0409091 A1
(43) Pub. Date: Dec. 29, 2022

(54) SYSTEMS AND METHODS FOR GAIT ANALYSIS

(71) Applicants: UNIVERSITY OF MARYLAND, COLLEGE PARK, College Park, MD (US); UNIVERSITY-INDUSTRY COOPERATION GROUP OF KYUNG HEE UNIVERSITY, Gyeonggi-do (KR)

(72) Inventors: Jae Kun SHIM, Clarksville, MD (US); Hyunji LEE, Yongin-si (KR); Yunjung HEO, Yongin-si (KR); Jumyung UM, Yongin-si (KR)

(21) Appl. No.: 17/778,890

(22) PCT Filed: Nov. 25, 2020
(86) PCT No.: PCT/US2020/062200

§ 371 Date: May 23, 2022

Related U.S. Application Data
(60) Provisional application No. 62/940,615, filed on Nov. 26, 2019.
(60) Provisional application No. 63/112,077, filed on Nov. 10, 2020.

(51) Int. Cl. A61B 5/103; A43B 13/14; A43B 3/34; A61B 5/00

(52) U.S. Cl. CPC A61B 5/1038; A43B 13/14; A43B 3/34; A61B 5/6807

(57) ABSTRACT

A gait analysis apparatus includes a bottom insole pad, a top insole pad layered over the bottom insole pad and configured to be worn by a limb of a subject, a plurality of force sensors configured to sense force exerted by the limb at least in two directions and affixed between the top insole pad and the bottom insole pad, and a processor configured to collect measurement data from the plurality of force sensors and determine a pose of or abnormality in the limb based on the measurement data and a predetermined profile.

### CROSS-REFERENCE TO RELATED APPLICATIONS

This application claims the benefit of U.S. Provisional Application Ser. No. 62/940,615 filed on Nov. 26, 2019, and U.S. Provisional Application Ser. No. 63/112,077 filed on Nov. 10, 2020. The entire contents of both applications are incorporated herein by reference.

### FIELD

This disclosure generally relates to systems and methods for gait analysis and, in particular, to systems and methods for detecting abnormalities in or a pose of a limb of a subject.

### BACKGROUND

Individuals with abnormal locomotion habits or problems confront difficulties in their daily lives. These abnormalities induce acute or chronic pain while walking, running, or using one or both of their feet. Ground reaction force (GRF) has been used to quantify mechanical interactions between the foot and the ground, and to calculate force and torque experienced by joints. Thus, GRF is an important factor to understand movements of the limb (e.g., a foot) of a subject because it is the only external force acting on a human body, except for gravity while in motion. Conventionally, force plates have been used to measure three dimensional (3-D) resultant GRF accurately.

Conventional methods of measuring GRF employ large, heavy, high-cost 6-degrees-of-freedom GRF device 100, as illustrated in FIG. 1. The GRF device 100 includes over-ground force plates 110a-110n anchored in laboratory environment, which is stationary.

The force plates 110a-110n generally measure forces exerted thereover while a subject person stands in a stance mode as shown in FIG. 1B, or walks in a walking mode as shown in FIG. 1C. The conventional GRF device 100 provides high reliability. While in the stance and walking modes, only one or two from among the force plates 110a-110n generate measurements while the others do not. Thus, even though the GRF device 100 provides highly reliable measurement data, the efficiency of the force plates 110a-110n is substantially low. Further, a duration of the measurement is limited based on the length of the conventional GRF device 100 and a walking speed. The duration is further limited in a running mode due to a greater speed than the walking speed. Thus, with these reasons, the GRF device 100 cannot be used to measure GRF in daily activities outside of a laboratory environment.

Many attempts have been made to scale down the technology or to implement wearable devices. Typically, wearable devices for estimating GRF are divided into two types: one is to attach sensors on the body, and the other is to insert or attach sensors inside shoes. Generally, information is collected by sensors or wearable devices and analyzed through software, and both hardware and software are commonly called a gait analysis system.

For example, some sensors, which are embedded within gait analysis systems, merely measure normal force distribution and do not allow measurement of force in the anterior-posterior direction or shear force, which is fundamental in measuring balance, velocity and acceleration of the movements, through the force interactions between the foot and the ground.

Accelerometers are attached on the subject's body to estimate GRF, inertial measurements unit (IMU) sensors are used to estimate walking or running GRF profiles, or uniaxial accelerometers are used to estimate walking or running GRF profiles. All of these can estimate GRF by attaching sensors on the body, but merely collect indirect data of GRF and cannot directly monitor dynamic forces between the foot and the ground.

Several force sensitive resistors (FSRs) have been used in a low-cost kinetic gait system insole. FSRs, however, are made specifically for each subject model and detect a subject's GRF and estimate moments from the knee and the ankle. The FSRs measure squatting and getting up from a sitting position and add to the weight shifting and walking motions. However, the low-cost kinetic gait system insoles pose fundamental limitations of a narrow sensing area and the fixed coordinate system.

In cases, a thick, bulky 3-axis force sensors have been attached on the surface on shoes and called as a mobile force plate. This device measures GRF, which is transformed its local coordinate system to a global coordinate system. Some other sensors have been used in gait analysis systems to measure 3-axis GRF. They, however, have provided GRF only at one point, but do not provide the resultant GRF or full GRF time profiles along the anterior-posterior direction or the moving direction.

Some methods have suggested comparing a weight shift between standing and moving (e.g., walking or running) positions with previous data. In particular, two methods have utilized nanocomposite piezo responsive foam sensors for estimating 3-D GRF. One was for inter-subject which set optimal variables for each subject and the other was for intra-subject which used a single data set for all subjects combined.

However, typical wearable devices do not measure critical variables, such as shear force, or do not accurately measure or determine the forces involved in locomotion. Further, gait analysis systems still need improvements so that other factors, which affect the performance thereof, such as velocity and the demographics of subjects, can be removed. Furthermore, the size of conventional gait analysis devices needs to be scaled down.

### SUMMARY

This disclosure generally relates to systems and methods for gait analysis using a gait analysis device, which can be worn by a foot of a subject so that gait analysis can be performed at any place other than in a laboratory environment and can provide many measurement points as needed.

Described herein is a gait analysis apparatus, which includes a bottom insole pad, a top insole pad layered over the bottom insole pad and configured to be worn by a limb of a subject, a plurality of force sensors configured to sense force exerted by the limb at least in two directions and affixed between the top insole pad and the bottom insole pad, and a processor configured to collect measurement data from the plurality of force sensors and determine a pose of or abnormality in the limb based on the measurement data and a predetermined profile.

In aspects, a top surface of the top insole pad is made of a non-slippery material.

In aspects, each force sensor is a piezo-resistive force sensor.

In aspects, the at least two directions include a superior-inferior direction and an anterior-posterior direction of the subject. Each force sensor is further configured to sense force in a direction perpendicular to the superior-inferior direction and to the anterior-posterior direction.

In aspects, a deep learning algorithm compares previous measurement data, which have been collected by the plurality of force sensors, synchronously with measurement data obtained from a force plate, and generates the predetermined profile.

In aspects, the plurality of force sensors are affixed at places where the limb presses substantially over the top insole pad. These places can include a first distal phalanx, metatarsal joints, and calcaneus of the limb.

In aspects, the measurement data is normalized based on a weight of the subject and a time span of a quiet standing phase with no or minimal movements.

In aspects, the gait analysis system further includes an amplifier configured to amplify analog signals from the plurality of force sensors. The amplified analog signals are digitized to generate the measurement data.

Further described herein is a gait analysis method for determining abnormality in a limb of a subject according to aspects of the present disclosure. The gait analysis method includes detecting places when the limb presses substantially over a stain pad, affixing a plurality of force sensors at these places in an insole pad, generating measurement data from the plurality of force sensors while the insole pad is worn by the limb, and comparing the measurement data from the plurality of force sensors with a predetermined profile to determine a pose of or abnormality in the limb.

In aspects, detecting the places includes placing the stain pad over a blank pad, and receiving a footprint of the limb, which has been stained on the blank pad by the stain pad. The places are detected based on the footprint stained on the blank pad.

In aspects, the places can include a first distal phalanx, metatarsal joints, and calcaneus of the limb.

In aspects, the insole pad includes a bottom pad and a top pad.

In aspects, a top surface of the insole pad is made of a non-slippery material.

In aspects, the plurality of force sensors are piezo-electric sensors.

In aspects, the predetermined model has been generated by a deep learning algorithm.

In aspects, the gait analysis method further includes normalizing the sensor data based on a weight of the subject and a time span of a stance phase. The stance phase begins when a heel of the limb strikes a ground and ends when a toe of the limb lifts off the ground.

In aspects, gait analysis method further includes amplifying analog signals from the plurality of force sensors. The amplified analog signals are digitized to generate the measurement data.

Further described herein is a non-transitory computer-readable medium including instructions thereon that, when executed by a computer, cause the computer to perform a gait analysis method for determining abnormality in a limb of a subject according to aspects of the present disclosure. The gait analysis method includes detecting places when the limb presses substantially over a stain pad, affixing a plurality of force sensors at the places in an insole pad, generating measurement data from the plurality of force sensors while the insole pad is worn by the limb, and comparing the measurement data from the plurality of force sensors with a predetermined profile to determine a pose of or abnormality in the limb.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

Various aspects are illustrated in the accompanying figures with the intent that these examples are not restrictive. It will be appreciated that for simplicity and clarity of the illustration, elements shown in the figures referenced below are not necessarily drawn to scale. Also, where considered appropriate, reference numerals may be repeated among the figures to indicate like, corresponding or analogous elements. The figures are listed below.

FIG. 1A is a block diagram of a conventional GRF detection device;

FIGS. 1B and 1C are graphical illustrations of a stance mode and a walking mode on the conventional GRF device of FIG. 1A;

FIG. 2 is a graphical diagram of a gait analysis apparatus according to various aspects of the present disclosure;

FIG. 3 is a graphical diagram of a force sensor of the gait analysis apparatus according to various aspects of the present disclosure;

FIG. 4 is a graphical illustration for detecting no load data for the gait analysis apparatus according to various aspects of the present disclosure;

FIGS. 5A-5C are graphical representations of measurement data according to various aspects of the present disclosure;

FIGS. 6A and 6B are graphical representations of measurement data with a GRF profile for walking according to various aspects of the present disclosure;

FIGS. 7A and 7B are graphical representations of measurement data with a GRF profile for running according to various aspects of the present disclosure;

FIG. 8 is a flowchart for a gait analysis method according to various aspects of the present disclosure; and

FIG. 9 is a block diagram of a computing device according to various aspects of the present disclosure.

### DETAILED DESCRIPTION

The present disclosure relates generally to a system and method for gait analysis. The systems and methods estimate real-time, resultant ground reaction force (GRF) by force sensors at multiple points inside of a shoe, where a foot exerts pressure, and estimate of 3-D GRF magnitude, direction, and application points may be determined. Based on these measurement data by the force sensors, a pose of or abnormalities in the foot can be determined by using artificial intelligence/machine learning related algorithms or methods. Further, a pose of the whole body of the subject may be predicted based on the measurement data.

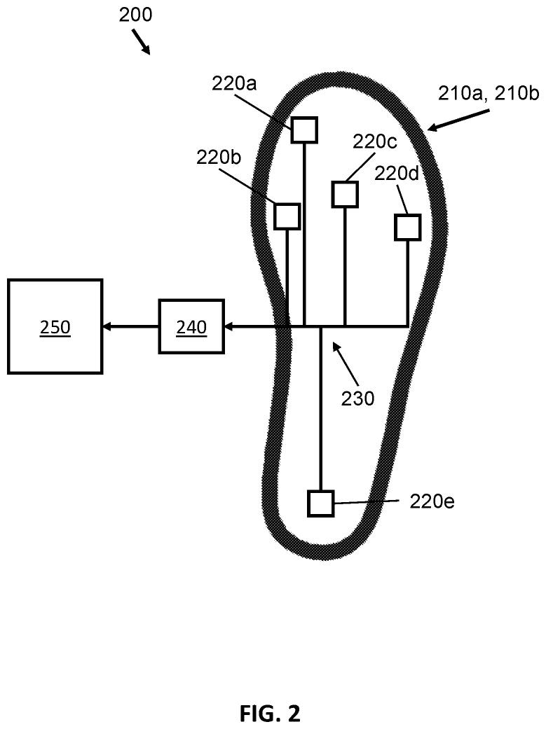

FIG. 2 shows a gait analysis apparatus 200 according to various aspects of the present disclosure. The gait analysis apparatus 200 may be installed in an insole 210 of a shoe as shown in FIG. 2, so that the gait analysis apparatus 200 may be wearable by a subject, thereby making measurements possible at any place besides a laboratory environment.

The gait analysis apparatus 200 may include force sensors 220a-220e configured to measure forces, in particular GRF, exerted thereon, a communication bus 230, an amplifier 240, and a computing device 250. The placement of the force sensors 210a-210e in the insole 210 may be designed to minimize disturbances from the subject's movements by positioning the force sensors 210a-210e to contact the sole of the subject's foot. The insole 210 of FIG. 2 is illustrated for the subject's right foot, but may be made for the subject's left foot.

To identify appropriate positions for the force sensors 210a-210e, a stain pad together with a blank pad may be worn by the subject's foot. The stain pad may be a carbon paper. The stain pad may be stapled at the edges thereof with the blank pad. When the subject makes a stance or movements on the pads, the stain is transferred to or trapped in the blank pad as in a form of footprint at places where the foot presses the most on the stain pad. In an aspect, these locations may be the first distal phalanx, first metatarsal joint, third metatarsal joint, fifth metatarsal joint, and calcaneus.

The force sensors 210a-210e may be accordingly affixed on the first distal phalanx, first metatarsal joint, third metatarsal joint, fifth metatarsal joint, and calcaneus of the subject's foot, respectively. The number of the force sensors 210a-210e is not limited to five but may be less than five. For example, the number of the force sensors 210a-210e may be three or two. For examples, the force sensors 210a-210e may be placed on the first distal phalanx, the calcaneus, and one of the first metatarsal, third metatarsal, and fifth metatarsal joints, or on the first distal phalanx and the calcaneus.

In an aspect, the number of the force sensors 210a-210e may be greater than five to further identify torques, inertia, movements, locations of joints or body parts of the subject.

The insole 210 may include one or more layers, for example, a top insole pad 210a and a bottom insole pad 210b. The force sensors 210a-210e may be affixed between the top and bottom insole pads 210a and 210b. In an aspect, the top insole pad 210a may be made of a non-slippery material or include a non-slippery top surface such that the subject's foot may not freely move on the top insole pad 210a and the force sensors 210a-210e may measure forces at the locations corresponding to the footprint obtained from the stain and blank pads.

To prevent movements of the bottom insole pad 210b when the gait analysis apparatus 200 is installed in a shoe, another pad, which is non-slippery, may be attached to the bottom of the bottom insole pad 210a. Further, to prevent movements of the force sensors 210a-210e between the top and bottom insole pads 210a and 210b, another preventive measure may be inserted around the force sensors 210a-210e and between the top and bottom insole pads 210a and 210b. The preventive measure may be a mesh sticker or adhesive.

The force sensors 210a-210e may measure forces exerted thereon and the measurement data is transferred to the amplifier 240 via a communication bus 230. The amplifier 240 may amplify the amplitude of the measurement data. The amplifier 240 may be an analog front-end, which includes filters to filter out noises from the measurement data.

In an aspect, the number of amplifier 240 may correspond to the number of the force sensors 210a-210e so that each amplifier 240 may amplify the analog signal generated by the corresponding force sensor.

The amplifier 240 may be connected to the computing device 250 via a wired connection or a wireless connection as the connection between the force sensors 210a-210e and the amplifier 240. The computing device 250 may receive and process the amplified sensor signal via a bus wire (e.g., universal serial bus (USB) or a micro USB). The computing device 250 may perform analysis on the measurement data, compare the analyzed data with a GRF profile, and determine a pose of the foot or abnormalities in the foot. In an aspect, the computing device 250 may transmit to an external computing device the measurement data via a wireless connection, which may be Bluetooth®, near field communication (NFC), WiFi™, or any other wireless protocols.

In an aspect, the computing device 250 may include an analog-to-digital converter (ADC), which convert the amplified analog signal into a digital signal. The digital signal may be in a form of hexadecimal or decimal.

In an aspect, the computing device 250 may be place on a top of the shoe or in any other places where the movements of the subject is not disturbed by the computing device 250, and powered by a battery, which may be placed on, for example, the ipsilateral side of the subject. The battery may be placed on any location, which does not affect the measurement data. In another aspect, the computing device 250 may be wirelessly powered by a remote power supply.

In an aspect, the computing device 250 may reset no-load voltage value of the force sensors 210a-210e and start collecting measurements data from the force sensors 210a-210e. Further, the computing device 250 may synchronously collect the measurement data from the force sensors 210a-210e at a sampling frequency, such as 100 Hz.

In another aspect, the computing device 250 may include a network interface, which is in a wired connection or wirelessly connected to an external computing device, which is not shown, and transmit the measurement data to the external computing device via the network interface, such as Bluetooth®, NFC, WiFi™, or any other communication protocols. The external computing device may control the computing device 250 to perform the functions/tasks of the gait analysis apparatus 200. A customized program may be employed to control the computing device 250.

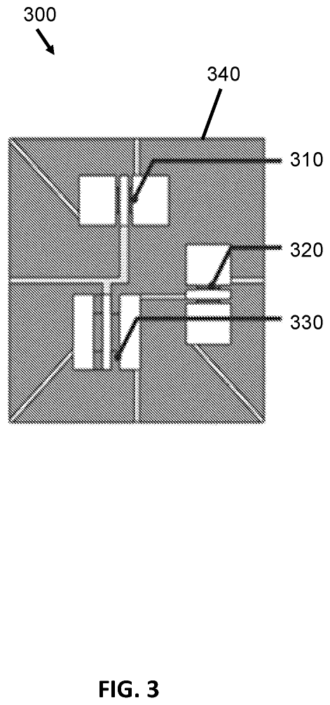

An example of each of the force sensors 210a-210e may be illustrated in FIG. 3 as a force sensor 300 according to aspects of the present disclosure. The force sensor 300 includes a substrate 340 and 3-axis force detectors affixed on the substrate 340.

Each three-axis force detector may be thin, lightweight 3-axis piezo-resistive force sensor, which calculates the force from the difference between the unloaded voltage and the loaded voltage. The 3-axis force detectors may include X-axis force detector 310, Y-axis force detector 320, and Z-axis force detector 330. The X-axis force detector 310 may detect a force along the medial-lateral direction or a direction perpendicular to a walking or running direction, the Y-axis force detector 320 may detect a force (shear force) along the anterior-posterior direction or the walking or running direction, and the Z-axis force detector 330 may detect a force along the superior-inferior direction or a direction normal to the ground.

In an aspect, the measurement data may vary depending on temperature. Thus, a manufacturer of the three-axis force detector may provide a coefficient matrix, which is used to calibrate the measurement data based on the temperature.

In an aspect, the force sensor 300 may be a 2-axis force sensor and include the Y-axis force detector 320 and the Z-axis force detector 330, when the force along the medial-lateral direction is negligible in determination of a pose of or abnormalities in the foot.

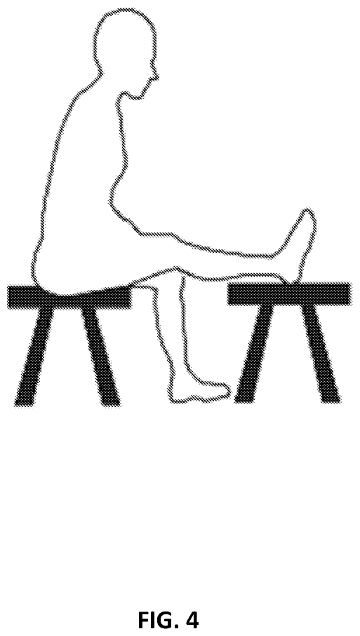

Prior to measurement of forces by the force sensors 210a-210e, no load data may be collected as shown in FIG. 4. While the gait analysis apparatus 200 is put on by the foot of the subject, the subject places the foot on a chair or a stool so that the foot is in a resting mode. The data collected during the resting mode is called as the no load data, which may be used as a reference or offset data to measure the actual force data.

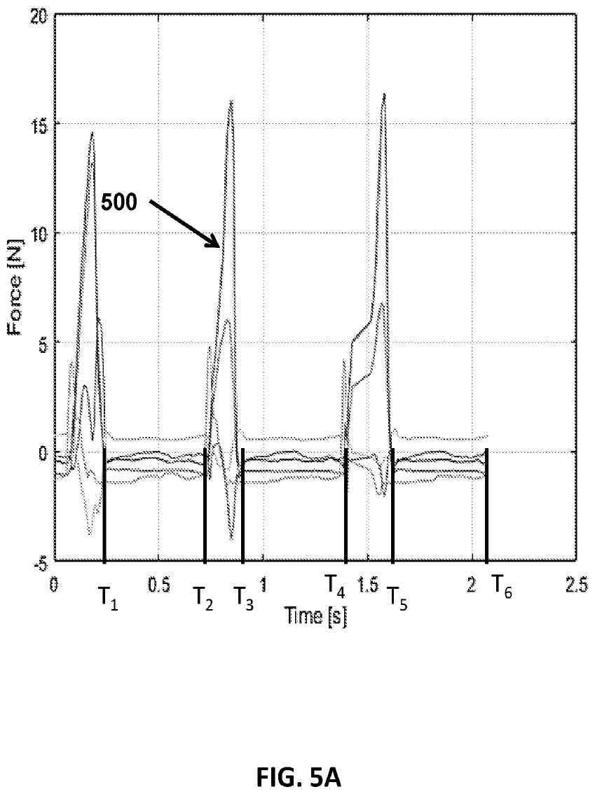

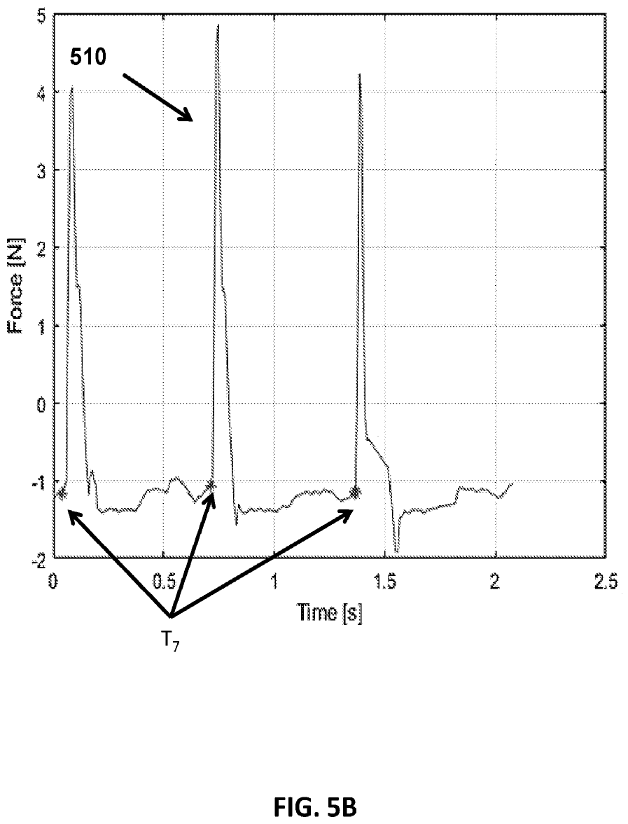

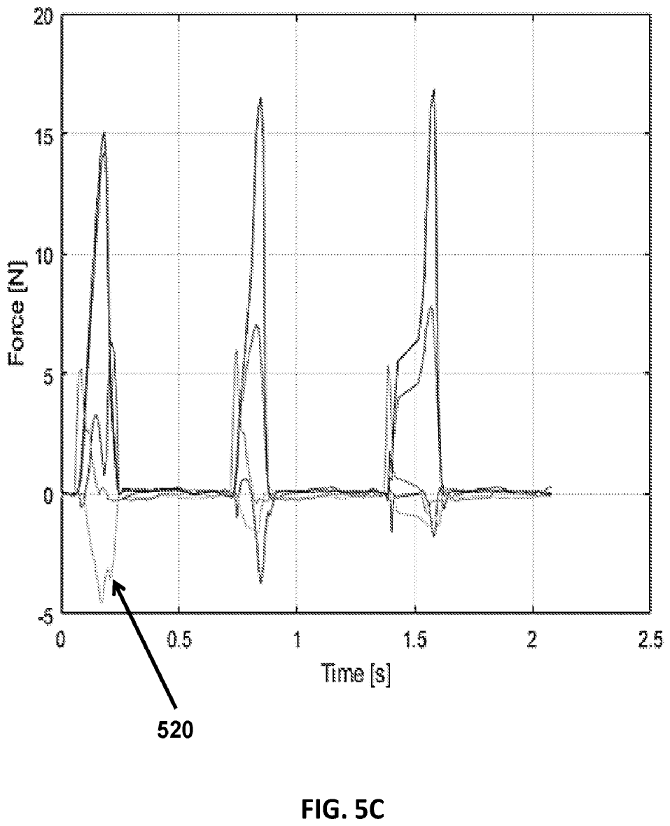

For example, FIG. 5A shows five raw data curves 500 from the force sensors 210a-210e, and FIG. 5C shows five adjusted data curves 520 after the offset removal. Five measurement curves of FIGS. 5A and 5C represent the measurement data from the force sensors 210a-210e while the subject makes six walking steps. Specifically, FIG. 5B shows measurement curve 510 from the force sensor 220e located in the calcaneus. The vertical axes of FIGS. 5A-5C represent force in unit of Newton, and the horizontal axes thereof represent times in second.

Since the gait analysis apparatus 200 is worn in only one foot of the subject, measurements data from the force sensors 210a-210e only show measurement data of three walking steps. From zero to T₁, the foot, which puts on the gait analysis apparatus 200, is in a stance phase meaning that the foot touches the ground, and, from T₁ to T₂, the foot is in a swing phase meaning that the foot is in the air. Likewise, during periods from T₂ to T₃ and from T₄ to T₅, the foot is in the stance phase, and during periods from T₃ to T₄ and from T₅ and T₆, the foot is in the swing phase. Conversely, the other foot is in the phase approximately opposite to the phase of the foot.

During the stance phase, the force sensors 210a-210e generate measurement data as shown in FIGS. 5A-5C. In consideration of the measurement curve 510 of FIG. 5B, there are three hikes in the measurement curve 510 starting from T₇, which represents a time when the heel of the foot touches the ground. Thus, the stance phase may start at T₇ when the heel touches the ground and ends when the first distal phalanx or big toe lifts off the ground.

On the other hand, during the swing phase, the force sensors 210a-210e generate no meaningful measurement data. Nevertheless, the force sensors 210a-210e generate some measurement data during the swing phases from T₁ to T₂, from T₃ to T₄, and from T₅ and T₆ as shown in FIG. 5A, which may represent the no load data. Thus, when the no load data or offset data is collected during the resting mode as shown in FIG. 4, the measurement data can be adjusted by removing the offset data therefrom. FIG. 5C shows the adjusted measurement data and no noticeable data can be shown during the swing phases from T₁ to T₂, from T₃ to T₄, and from T₅ and T₆.

Generation of GRF Profile

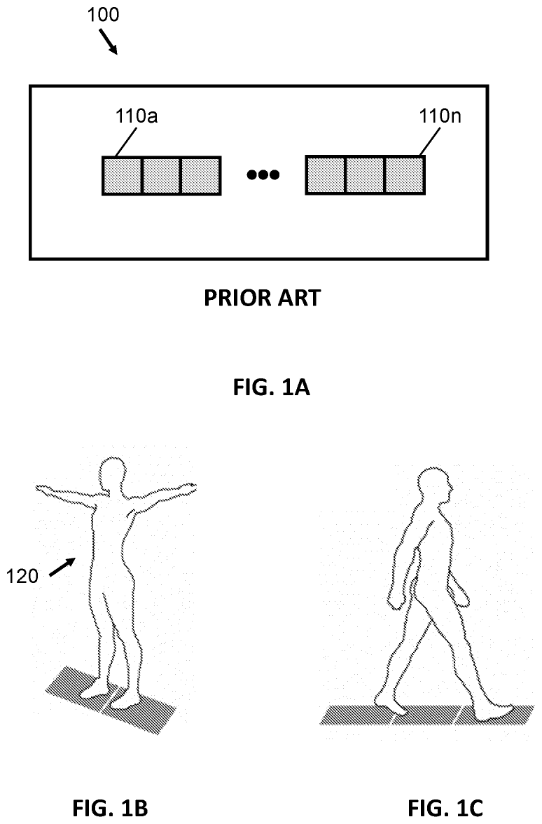

To determine a pose or abnormalities in the foot, a reference profile or a GRF profile is needed. The GRF profile may be generated by using the conventional GRF device 100 of FIG. 1 and the gait analysis apparatus 200 of FIG. 2 together. While the subject puts on the gait analysis apparatus 200 of FIG. 2, the subject may stand, walk, or run on the conventional GRF device 100. The conventional GRF device 100 may generate measurement data at 1000 hertz (Hz), while the force sensors 210a-210e may generate measurement data at 100 Hz.

The force sensors 210a-210e and the force plates 110a-110n may synchronously generate measurement data in analog form or measurement signals. A lowpass filter may be used to filter out low frequencies from the measurement signals. For example, the cut-off frequency of the lowpass filter may be 20 Hz and the lowpass filter may be a butter-worth filter.

After removing the low frequency parts from the measurement signals, the measurement data from the force sensors 210a-210e may be up-sampled to match the sampling frequency or the fixed length of the measurement data from the force plates 110a-110n. Up-sampling may mean that velocity of the subject is ignored. Nevertheless, by reducing the velocity factor, the GRF profile may be obtained in a faster phase and with a fewer measurement data than with the velocity factor. In an aspect, the length of the measurement data may be greater or smaller than 1024 or equal to 1024.

Further, the measurement data from both the force sensors 210a-210e and the force plates 110a-110n may be normalized by dividing by the weight of the subject, thereby reducing the weight factor.

Since the conventional GRF device 100 generates precise and accurate measurement data, the measurement data from the gait analysis apparatus 200 are processed to mimic or assimilate the measurement data from the conventional GRF device 100. In this way, need for the conventional GRF device 100 is removed and the gait analysis apparatus 200 may generate measurement data as accurate as the conventional GRF device 100.

Along advancement in artificial intelligence (AI) technology, many gait analysis researchers utilizes machine learning and deep learning. For example, a linear regression model has been used for specific subjects and needed pre-processing on complex gait data, and neural network models have given chances to overcome the limitation of existing methods. The neural network models have been capable of faster, more various results from gait databases that are constantly growing in volume and range.

Feed forward neural network or multilayer perception (MLP) models have predicted 3-D GRF based on accelerometer data with one hidden layer and three output layers.

Artificial neural network (ANN) models have been applied to predict 3-D GRF using lower body kinematics. To utilize the ANN models, professional athletes' force and motion capture running data are collected with respect to different speeds, where acceleration of the shanks data is used as input data and the predicted GRF shows low root mean square errors under 0.2 body weights (BW).

According to the present disclosure, measurement data from the gait analysis apparatus 200 for many subjects are collected and processed by using AI algorithms as described above to generate a GRF profile. In particular, the AI algorithms may include multilayer perception (MLP) model, deep MLP model, and one dimensional (1-D) convolutional neural network (CNN) model. The AI algorithms are not limited to this list but can include others as readily apprehended by a person having ordinary skill in the art.

The MLP model may include multiple layers, which are fully connected by hyperbolic or ReLU tangent functions. The deep MLP may provide a better result than the MLP because the deep MLP may include all layers in the MLP and include additional layers into the hidden layers in the MLP. For example, more layers in the hidden layers may result in latent variables, which are then decoded to the output layer. Due to the added layers, the deep MLP may perform better than the MLP. The size of the layers in the deep MLP is getting smaller first and then getting bigger.

The 1-D CNN model may include an input layer, convolutional layers, and an MLP layer. The convolutional layers of 1-D CNN model may convolute the input layer to generate pooling layers by max pooling so that most influential features can be down sampled. Specifically, the first convolution layer may merge single or multiple sensor measurement data into one single data. The measurement data may be compressed into a fixed length data. Back-propagate neural network may reduce the gap with feed-back of adjusting the weight factor of each link between network nodes. In an aspect, to keep the shape of GRF profile crossing minus and plus, self-gated activation functions (Swish) may be applied to a GRF regression neural network via x*sigmoid (beta*x), where the sigmoid function S(x) is 1/1+e^{-x}, and beta is a constant.

By performing AI algorithms, a GRF profile may be generated for running or walking. After being normalized by dividing the measurement data by the weight of the subject, the GRF profile may be applied to each subject regardless of the weight of the subject. In other words, the weight is independent from the GRF profile. In this way, other factors including sex, age, height, velocity, or any other individual features may be also considered in the GRF profile, thereby such features being made independent from the GRF profile.

In an aspect, the GRF profile may be applied in the fields of medicine, biomechanics, motor control, and robotics. In another aspect, real-time GRF profiles may provide foot-ground force interaction in human locomotion, help control exoskeletons or other robots, improve sports performance, help prevention of injuries, and promote rehabilitation for the impaired or the injured. Motions of robots may be programmed by following the GRF profiles for running, walking, or standing, sitting, or any other motions so that the robots can move like a human being.

In another aspect, the GRF profile may be generated for walking, running, jumping, standing, hopping, fast walking, fast running, jump-roping, golf-swing, etc. For explaining purposes only, two GRF profiles are described below for walking and running.

In a further aspect, the GRF profile may be used in locomotive disabilities, such as Parkinson's or stroke, to control a prosthesis, and to train athletes.

Walking Mode

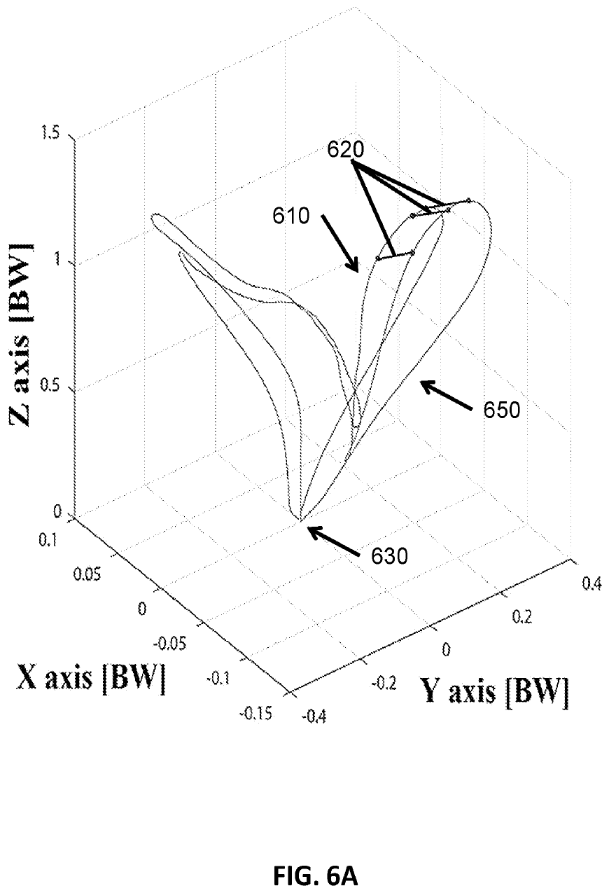

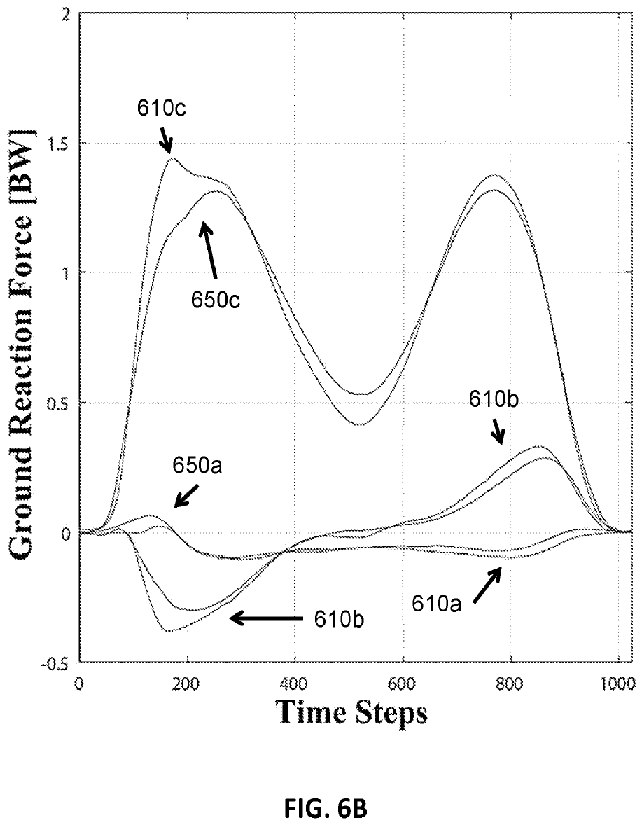

FIGS. 6A and 6B show a GRF profile with sensor measurement data during a walking mode according to aspects of the present disclosure. In particular, FIG. 6A shows a GRF profile curve 610 and sensor measurement curve 650 in a 3-D space. The X-, Y-, and Z-axes include a unit, which is adjusted based on the weight of the subject. In other words, the unit of the three axes is newton (N) divided by kilogram (kg) according to International System of Units (SI). In an aspect, when other normalization variables are incorporated into the measurement data, the unit of the three axes may be correspondingly changed.

The GRF profile curve 610 and the measurement curve 650 are made by time-series data. Three lines 620 connecting the GRF profile curve 610 and the measurement curve 650 may represent differences between two curves at the corresponding time. Since the origin 630 is the starting point for the GRF profile curve 610 and the measurement curve 650, the corresponding time may be measured from the origin 630.

In an aspect, the sum of the differences between the GRF profile curve 610 and the measurement curve 650 at each corresponding time may be used to determine a pose of or abnormalities of the foot. A root mean square (RMS), normalized RMS, or any other measures may be also used.

In another aspect, a fitting difference (FIT) may be used to calculate fitting accuracy between the GRF profile and the measurement data in a matrix form. R squared is the square of the correlation between the measurement data and the GRF profile and the goodness of fit statistics can be evaluated. The FIT between the measurement data and the GRF profile may be calculated in two ways: each of X-, Y-, and Z-axes in one dimension and whole direction at the same time in three dimensions. 3-D FIT may be used to reduce the type 1 error when the GRF profile is multi-dimensional information. The 1-D or 3-D FIT [%] between the measurement data and the GRF profile may be calculated by substituting the matrix expressions according to the dimension.

$$3 - {DGRF}_{j = 1} = [ \begin{matrix}{X}_{( 1 , j = 1 )} & {Y}_{( 1 , 1 )} & {Z}_{( 1 , 1 )} \\ ⋮ & ⋱ & ⋮ \\ {X}_{( 1024 , j = 1 )} & {Y}_{( 1024 , 1 )} & {Z}_{( 1024 , 1 )}\end{matrix} ] \\ {FIT}_{j} [ \% ] = ( 1 - \frac{{SSE}_{j}}{{SST}_{j}} ) \times 100 where \\ {SSE}_{j} = NORM ( {GRF}_{{measurement}_{j}} - {GRF}_{{predicted}_{j}} ) and \\ {SST}_{j} = NORM ( {GRF}_{{measurement}_{j}} - \overline{{GRF}_{{measurement}_{j}}} ) \\ 1 - DGRF ( X axis ) = [ \begin{matrix}{X}_{( 1 , 1 )} & {X}_{( 1 , j )} & {X}_{( 1 , n )} \\ ⋮ & ⋱ & ⋮ \\ {X}_{( 1024 , 1 )} & {X}_{( 1024 , j )} & {X}_{( 1024 , n )}\end{matrix} ] \\ FIT [ \% ] = ( 1 - \frac{SSE}{SST} ) \times 100 where \\ SSE = NORM ( {GRF}_{measurement} - {GRF}_{predicted} ) and \\ SST = NORM ( {GRF}_{measurement} - \overline{{GRF}_{measurement}} ) \\ ( {X}_{ij} , i = Time steps , j = Test data number , \\ n = Total number of test data )$$

where X(i,j=1), Y(i,l), and Z(i,l) represents the i-th measurement data by the first force sensor along the X-, Y-, and Z-axes, NORM(x-y) returns the Euclidean norm or generally a distance between two matrixes x and y, GRFₘₑₐₛᵤᵣₑₘₑₙₜⱼ represents the j-th measurement data, G̅R̅F̅ₘ̅ₑ̅ₐ̅ₛ̅ᵤ̅ᵣ̅ₑ̅ₘ̅ₑ̅ₙ̅ₜ̅ⱼ̅ represents an average matrix of the j-th measurement data, GRF_{predictedj} represents the j-th predicted data based on the GRF profile, GRFₘₑₐₛᵤᵣₑₘₑₙₜ represents the measurement data, G̅R̅F̅ₘ̅ₑ̅ₐ̅ₛ̅ᵤ̅ᵣ̅ₑ̅ₘ̅ₑ̅ₙ̅ₜ̅ represents an average matrix of the measurement data, and GRF_{predicted} represents the predicted data based on the GRF profile.

To calculate the accuracy differences with each force sensor, the average of the 3-D FIT may be calculated. One-Way repeated-measures analysis of variance (ANOVA) may be used to test differences between the number conditions. Whole sensor combinations may be categorized as groups by the number of the force sensors. For example, group two means that the sensor combinations consist of the two locations from five force sensors. The average of FIT in each group may be dependent variables.

FIG. 6B illustrates the GRF profile curve 610 and the measurement curve 650 along the X-, Y-, and Z-axes according to aspects of the present disclosure. The GRF profile curve 610 is separated into 610a along the X-axis or the medial-lateral direction of the subject, 610b along the Y-axis or the anterior-posterior direction, and 610c along the Z-axis or the superior-inferior direction. Likewise, the measurement curve 650 is also separated into 650a, 650b, and 650c along the X-, Y-, and Z-axes, respectively. The vertical axis of FIG. 6B represents the body weight-adjusted GRF and the horizontal axis represents time stamps. 1-D FIT may be calculated from 610a and 650a, 610b and 650b, and 610c and 650c for each direction.

Table 1 below shows examples of 1-D and 3-D FITs in percentage.

TABLE 1
1-D and 3-D FIT results of MLP, Deep MLP, and 1-D CNN

| FIT [%] (Avg ± std) | MLP | Deep MLP | 1-D CNN |
|---|---|---|---|
| Z axis (1-D) | 66.67 ± 14.05 | 70.03 ± 13.45 | 76.94 ± 10.69 |
| Y axis (1-D) | 50.72 ± 40.91 | 64.08 ± 30.53 | 67.32 ± 31.72 |
| X axis (1-D) | −15.70 ± 66.68 | 0.92 ± 51.49 | 34.58 ± 31.33 |
| 3-axes (3-D) | 65.46 ± 13.86 | 69.76 ± 13.45 | 76.57 ± 10.60 |

As shown in Table 1, 1-D CNN provides best results. However, as described above, any other AI algorithms may be employed to generate the GRF profile so that intended results can be obtained.

Two convex portions or local maximums in 610c and 650c may represent moments of the GRF peaks in the vertical direction during braking and propulsion phases of walking, and one concave portion or the local minimum in 610c and 650c may represent a moment of transition between the braking phase and the propulsion phase. Differently, 610b and 650b have one concave portion or local minimum representing the moment of the GRF peak in the direction opposite to walking direction, and one convex portion or the local maximum representing the GRF peak in the direction of walking direction. Since the measurement curves 650a-650c closely follow the GRF profile curves 610a-610c for walking, the pose of the subject may be determined as walking. When the difference between the measurement curves 650a-650c and the corresponding GRF profile curves 610a-610c is greater than a predetermined threshold, the pose of the subject may be determined as non-walking or as having other movements. In an aspect, the shape of the GRF profile curves 610a-610c may be determined based on the local maximums and local minimums.

The magnitudes of the measurement curve 650a along medial-lateral direction are substantially small compared to those from the measurement curves 650b and 650c in determining a pose or abnormalities. Thus, in an aspect, the force sensors for determining walking may include 2-axis force sensor rather than the 3-axis force sensor as shown in FIG. 3. The 2-axis may measure forces along the Y-axis or the anterior-posterior direction and the Z-axis or the superior-inferior direction.

In another aspect for determining walking, the number of force sensors positioned in the metatarsal joints of the foot may be decreased from three as shown in FIG. 2 to one or even to zero. In other words, the gait analysis apparatus 200 may have the force sensors located only at the first distal phalanx and the calcaneus, or only at the first distal phalanx, the calcaneus, and one of the first, third, and fifth metatarsal joints.

Running Mode

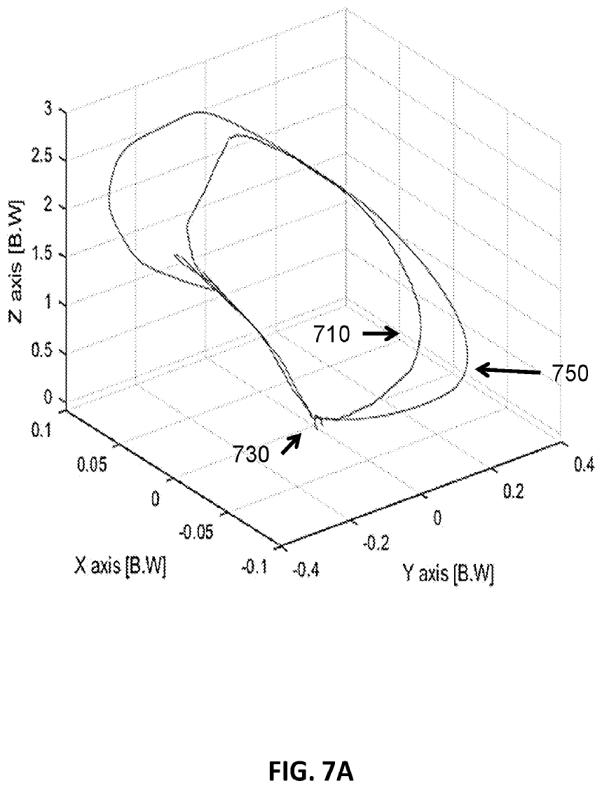

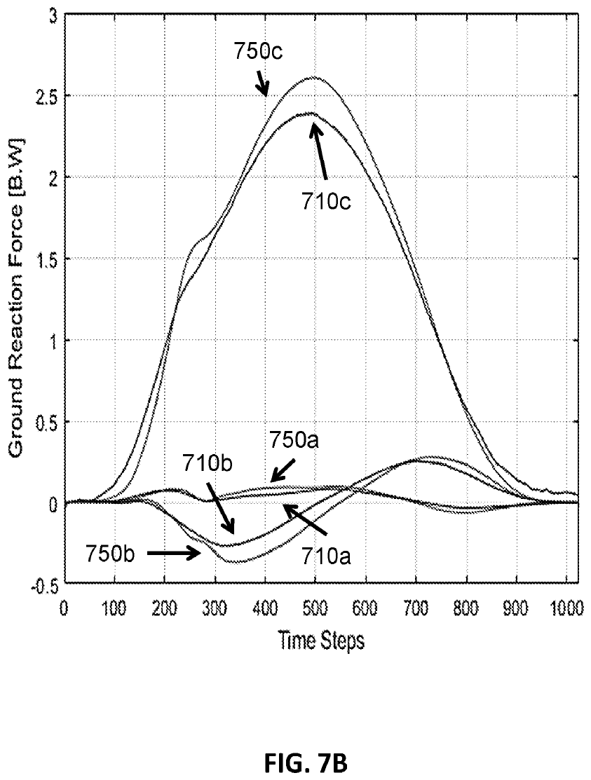

FIGS. 7A and 7B show a GRF profile curve 710 and measurement curve 750 during a running mode according to aspects of the present disclosure. In particular, FIG. 7A shows a GRF profile curve 710 for running and a measurement curve 750 in a 3-D space. The X-, Y-, and Z-axes include a unit, which is adjusted based on the weight of the subject as in FIG. 6A.

In the running mode, as the upper body becomes more inclined toward the front side, the position of the foot in the shoe changes. The change of foot position in the shoe is substantially different from that in the walking mode. Thus, the positions for the force sensors 210a-210e of FIG. 2 may be reconsidered and replaced with new positions based on a new footprint obtained from the stain and blank pads.

The GRF profile curve 710 and the measurement curve 750 are made by time-series data. Since the origin 730 is the starting point for the GRF profile curve 710 and the measurement curve 750, the corresponding time may be measured from the origin 730. In an aspect, the sum of the differences between the GRF profile curve 710 and the measurement curve 750 at each corresponding time may be used to determine a pose of or abnormalities of the foot. A root mean square (RMS), normalized RMS, or any other measures may be also used for the running mode.

FIG. 7B illustrates the GRF profile curve 710 and the measurement curve 750 along the X-, Y-, and Z-axes according to aspects of the present disclosure. The GRF profile curve 710 is separated into 710a along the X-axis or the medial-lateral direction of the subject, 710b along the Y-axis or the anterior-posterior direction, and 710c along the Z-axis or the superior-inferior direction. Likewise, the measurement curve 750 is also separated into 750a, 750b, and 750c along the X-, Y-, and Z-axes, respectively.

One convex portion of the local maximum in 710c and 750c signifies that the GRF profile is for running rather than walking. Differently, 710b and 750b have one concave portion or local minimum representing the moment of the GRF peak in the direction opposite to running direction, and one convex portion or the local maximum representing the GRF peak in the direction of running direction. Since the measurement curves 750a-750c closely follow the GRF profile curves 710a-710c for running, the pose of the subject may be determined as running. When the difference between the measurement curves 750a-750c and the GRF profile curves 710a-710c is greater than a predetermined threshold, the pose of the subject may be determined as non-running or as having other movements in the subject.

The magnitudes of the measurement curve 750a along medial-lateral direction are substantially small compared to those of the measurement curves 750b and 750c in determining a pose or abnormalities. Thus, in an aspect, the force sensors for determining running may include 2-axis force sensor rather than the 3-axis force sensor as shown in FIG. 3. The 2-axis may be the Y-axis or the anterior-posterior direction and the Z-axis or the superior-inferior direction because the magnitude of the measurement data along the X-axis or the medial-lateral direction is substantially small compared to the measurement data along the Y- and Z-axes.

In another aspect for determining running, the number of force sensors positioned in the metatarsal joints of the foot may be decreased from three as shown in FIG. 2 to one or even to zero. In other words, the gait analysis apparatus 200 may have the force sensors located only at the first distal phalanx and the calcaneus, or only at the first distal phalanx, the calcaneus, and one of the first, third, and fifth metatarsal joints.

Other Applications

The measurement data from the force sensors 210a-210e may be used to assimilate data of body or body segment positions obtained by motion capture systems and inertial sensors attached to subject's body or body segments. In this regard, body or body segment position data from motion capture systems and inertial sensors, which are capable of calculating torque or force at joints of the body in combination with GRF data, may be collected while the force sensors 210a-210e generate measurement data. Thus, the measurement data from the force sensors 210a-210e can be used to predict or determine forces or torques at joints (ankle, knee, hip, etc.) of the body.

In an aspect, the forces or torques at each joint may be predicted based on the measurement data from the force sensors. To do such, the number of the force sensors may be greater than five. After predicting the forces or torques at each joint, it is possible to predict full body poses or motions based on the measurement data from the force sensors. Further, the subject's foot pressure distribution or center of pressure position, motions and the spatial-temporal parameters may also be predicted based on the measurement data from the force sensors.

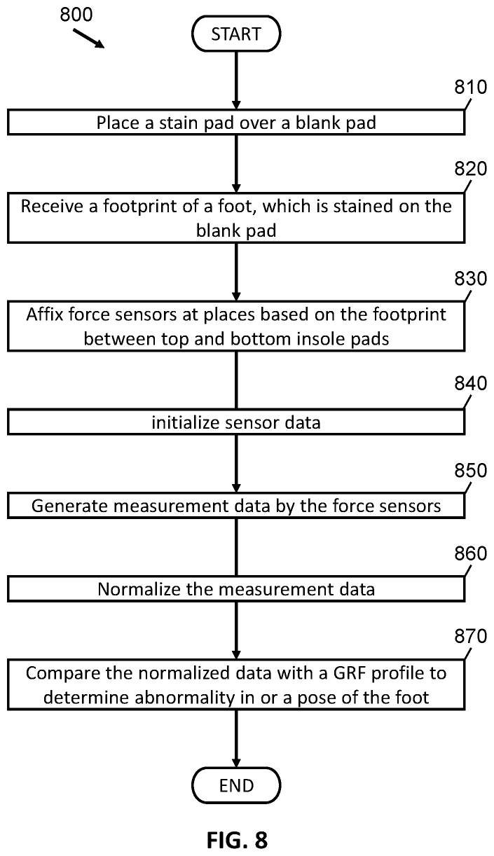

FIG. 8 shows a flowchart illustrating a method 800 for determining a pose of or abnormalities in a subject according to aspects of the present disclosure. The method 800 starts by identifying locations for force sensors in steps 810-830 and ends by comparing the sensor measurement data with a GRF profile in steps 850 and 860.

Specifically, in step 810, a stain pad is placed over a blank pad. The combination of the stain and blank pads is installed or positioned in a shoe of a subject. When the subject puts on the shoe and makes standing, walking, or running motions, the force exerted on the shoe leaves a footprint on the blank pad.

In step 820, the footprint of the foot is left on the blank pad due to the stain transferred from the stain pad. The footprint may include stained spots indicating the locations where the foot pushes more than any other places on the blank pad.

Based on the footprint, force sensors may be affixed in an insole in step 830. The insole may include top and bottom insole pads and the force sensors may be affixed between the top and bottom insole pads. In an aspect, the top surface of the top insole pad may be made of a non-slippery material. In another aspect, the force sensors may be thin, lightweight 2-axis or 3-axis piezo-resistive force sensors, which calculate the force from the difference between the unloaded voltage and the loaded voltage.

In step 840, the sensor measurement data may be initialized. Initialization of the sensor measurement data may include collecting no load data or offset data when the subject takes a resting position with the force sensor on the foot, and removing the no load data off from measurement data.

When the subject makes a walking or running movement, the force sensors generate measurement data in the step 850. To increase accuracy and reliability of the method 800, the measurements are to be made for at least two or more cycles of walking or running movement.

The measurement data may be normalized in step 860. The body weight of the subject may divide the measurement data so that the body weight can be removed from the normalized measurement data. In an aspect, other factors such as preferred walking speed or running speed may be taken into consideration in the normalization step.

In step 870, the normalized measurement data is compared with a GRF profile to determine abnormalities in or a pose of the foot of the subject. The GRF profile for running may be different from a GRF profile for walking. If the normalized measurement data is close to the GRF profile for running or walking, the pose of the subject may be running or walking, respectively. If the normalized measurement data has a difference from the GRF profile greater than a threshold, it is determined that there are abnormalities in the body or foot of the subject.

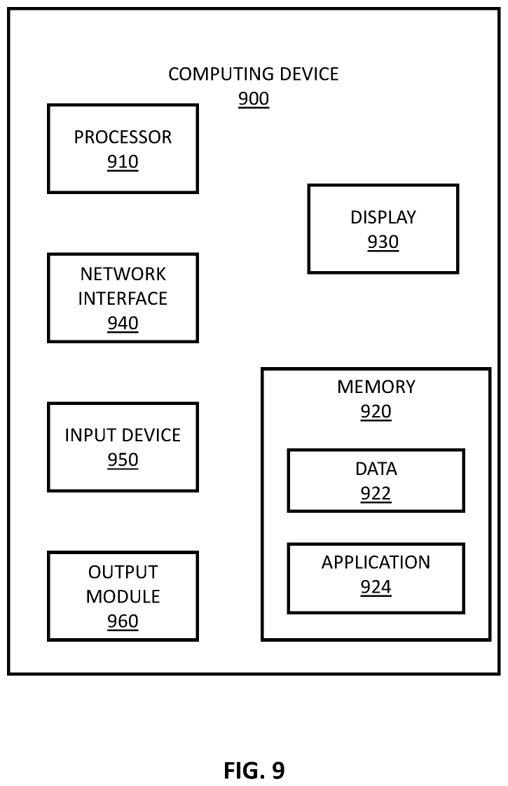

Turning now to FIG. 9, a block diagram is provided for a computing device 900, which can be the computing device 250 of FIG. 2. The computing device 900 may include a processor 910, a memory 920, a display 930, a network interface 940, an input device 950, and/or an output module 960. The memory 920 may include any non-transitory computer-readable storage media for storing data and/or software that is executable by the processor 910 and which controls the operation of the computing device 900.

In an aspect, the memory 920 may include one or more solid-state storage devices such as flash memory chips. Alternatively, or in addition to the one or more solid-state storage devices, the memory 920 may include one or more mass storage devices connected to the processor 910 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 910. That is, computer readable storage media may include non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, Blu-Ray or other optical storage, magnetic cassettes, magnetic tape, magnetic disk storage or other magnetic storage devices, or any other medium which can be used to store the desired information and which can be accessed by the computing device 900.

The memory 920 may store application 924 and/or data 922 (e.g., GRF profile and measurement data from the force sensors 220a-220e). The application 924 may, when executed by processor 910, perform gait analysis on the measurement data and compare the results of the gait analysis with the GRF profile using AI modules described above. In an aspect, the application 924 will be a single software program having all of the features and functionality described in the present disclosure. In another aspect, the application 924 may be two or more distinct software programs providing various parts of these features and functionality. Various software programs forming part of the application 924 may be enabled to communicate with each other and/or import and export various settings and parameters relating to the identification of a pose or abnormalities in the foot of subjects. The application 924 communicates via a user interface to present visual interactive features to the user on the display 930. For example, the graphical illustrations may be outputted to the display 930 to present graphical illustrations as shown in FIGS. 5A-7B.

The application 924 may include a sequence of process-executable instructions, which can perform any of the herein described methods, programs, algorithms or codes, which are converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, C, Delphi, Fortran, Java, JavaScript, machine code, operating system command languages, Pascal, Perl, PL1, scripting languages, Visual Basic, meta-languages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

The processor 910 may be a general purpose processor, a specialized graphics processing unit (GPU) configured to perform specific graphics processing tasks or parallel processing while freeing up the general purpose processor to perform other tasks, and/or any number or combination of such processors, digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

The display 930 may be touch-sensitive and/or voice-activated, enabling the display 930 to serve as both an input and output device. Alternatively, a keyboard (not shown), mouse (not shown), or other data input devices may be employed. The network interface 940 may be configured to connect to a network such as a local area network (LAN) consisting of a wired network and/or a wireless network, a wide area network (WAN), a wireless mobile network, a Bluetooth network, and/or the internet.

For example, the computing device 900 may receive, through the network interface 940, measurement data for the force sensors 220a-220e of FIG. 2, for example, which is time-series data in a stance, walking, or running mode. The computing device 900 may receive updates to its software, for example, the application 924, via the network interface 940. The computing device 900 may also display notifications on the display 930 that a software update is available.

The input device 950 may be any device by means of which a user may interact with the computing device 900, such as, for example, a mouse, keyboard, voice interface, or the force sensors 220a-220e of FIG. 2. The output module 960 may include any connectivity port or bus, such as, for example, parallel ports, serial ports, universal serial busses (USB), or any other similar connectivity port known to those skilled in the art. In an aspect, the application 924 may be installed directly on the computing device 900 or via the network interface 940. The application 924 may run natively on the computing device 900, as a web-based application in a cloud via the network interface 940, or any other format known to those skilled in the art.

The embodiments disclosed herein are examples of the disclosure and may be embodied in various forms. Although certain embodiments herein are described as separate embodiments, each of the embodiments herein may be combined with one or more of the other embodiments herein. It should also be understood that, depending on the example, certain acts or events of any of the processes or methods described herein may be performed in a different sequence, may be added, merged, or left out altogether (e.g., all described acts or events may not be necessary to carry out the techniques). In addition, Specific structural and functional details disclosed herein are not to be interpreted as limiting, but as a basis for the claims and as a representative basis for teaching one skilled in the art to variously employ the present disclosure in virtually any appropriately detailed structure.

What is claimed is:

1. A gait analysis apparatus comprising:
a bottom insole pad;
a top insole pad layered over the bottom insole pad and configured to be worn by a limb of a subject;
a plurality of force sensors configured to sense force exerted by the limb at least in two directions and affixed between the top insole pad and the bottom insole pad; and
a processor configured to collect measurement data from the plurality of force sensors and determine a pose of or abnormality in the limb based on the measurement data and a predetermined profile.

2. The gait analysis apparatus according to claim 1, wherein a top surface of the top insole pad is made of a non-slippery material.

3. The gait analysis apparatus according to claim 1, wherein each force sensor is a piezo-resistive force sensor.

4. The gait analysis apparatus according to claim 1, wherein the at least two directions include a superior-inferior direction and an anterior-posterior direction of the subject.

5. The gait analysis apparatus according to claim 4, wherein each force sensor is further configured to sense force in a direction perpendicular to the superior-inferior direction and to the anterior-posterior direction.

6. The gait analysis apparatus according to claim 1, wherein a deep learning algorithm compares previous measurement data, which has been collected by the plurality of force sensors, synchronously with measurement data obtained from a force plate, and generates the predetermined profile.

7. The gait analysis apparatus according to claim 1, wherein the plurality of force sensors are affixed at places where the limb presses substantially over the top insole pad.

8. The gait analysis apparatus according to claim 7, wherein the places are a first distal phalanx, metatarsal joints, and calcaneus of the limb.

9. The gait analysis apparatus according to claim 1, wherein the measurement data is normalized based on a weight of the subject and a time span of a quiet standing phase with no or minimal movements.

10. The gait analysis apparatus according to claim 1, further comprising:
an amplifier configured to amplify analog signals from the plurality of force sensors,
wherein the amplified analog signals are digitized to generate the measurement data.

11. A gait analysis method for determining abnormality in a limb of a subject, the gait analysis method comprising:
detecting places when the limb presses substantially over a stain pad;
affixing a plurality of force sensors at the places in an insole pad;
generating measurement data from the plurality of force sensors while the insole pad is worn by the limb; and
comparing the measurement data from the plurality of force sensors with a predetermined profile to determine a pose of or abnormality in the limb.

12. The gait analysis method according to claim 11, wherein detecting the places includes:
placing the stain pad over a blank pad; and
receiving a footprint of the limb, which has been stained on the blank pad by the stain pad,
wherein the places are detected based on the footprint stained on the blank pad.

13. The gait analysis method according to claim 11, wherein the places are a first distal phalanx, metatarsal joints, and calcaneus of the limb.

14. The gait analysis method according to claim 11, wherein the insole pad includes a bottom pad and a top pad.

15. The gait analysis method according to claim 11, wherein a top surface of the insole pad is made of a non-slippery material.

16. The gait analysis method according to claim 11, wherein the plurality of force sensors are piezo-electric sensors.

17. The gait analysis method according to claim 11, wherein the predetermined model has been generated by a deep learning algorithm.

18. The gait analysis method according to claim 11, further comprising:
normalizing the sensor data based on a weight of the subject and a time span of a quiet standing phase with no or minimal movements.

19. The gait analysis method according to claim 18, further comprising:
amplifying analog signals from the plurality of force sensors,
wherein the amplified analog signals are digitized to generate the measurement data.

20. A non-transitory computer-readable storage medium including instructions thereon that, when executed by a computer, cause the computer to perform a gait analysis method for determining abnormality in a limb of a subject, the gait analysis method comprising:
detecting places where the limb presses substantially over a stain pad;
affixing a plurality of force sensors at the places in an insole pad;
generating measurement data from the plurality of force sensors while the insole pad is worn by the limb; and
comparing the measurement data from the plurality of force sensors with a predetermined profile to determine a pose of or abnormality in the limb.
